(19) United States
(12) Patent Application Publication
(10) Pub. No.: US 2025/0114112 A1
(43) Pub. Date: Apr. 10, 2025

(54) OCCLUSION CROSSING CATHETERS AND METHODS FOR USING THE SAME

(71) Applicant: Bard Peripheral Vascular, Inc., Franklin Lakes, NJ (US)

(72) Inventors: Ruben RAMIREZ, Mesa, AZ (US); Aseem SINGH, Tempe, AZ (US); Amanda YOUNG, Phoenix, AZ (US)

(73) Assignee: Bard Peripheral Vascular, Inc., Franklin Lakes, NJ (US)

(21) Appl. No.: 18/834,791

(22) PCT Filed: Feb. 15, 2022
(86) PCT No.: PCT/US2022/016380

§ 371 Date: Jul. 31, 2024

(51) Int. Cl. A61B 17/22; A61B 17/00; A61B 17/3207

(52) U.S. Cl. CPC A61B 17/22012; A61B 17/00234; A61B 17/320725; A61B 2017/00137; A61B 2017/00238; A61B 2017/00305; A61B 2017/22017; A61B 2017/22038; A61B 2017/22079; A61B 2017/22094; A61B 2217/005

(57) ABSTRACT

The present disclosure relates to ultrasonic catheters including vibration wires that are positionable in a retracted position and an extended position, and their methods of use. In operation, catheters according to the present disclosure are operable in an occlusion crossing mode, in which the catheter emits ultrasonic energy with the vibration wires in the retracted position and in an atherectomy mode, in which the catheter emits ultrasonic energy with the vibration wires in the extended position, causing the vibration wires to oscillate. The vibration wires may directly or indirectly engage plaque within an artery, causing the plaque to break free from the artery wall. The oscillation of the vibration wires may be less abrasive than conventional cutting elements used in atherectomy procedures, such that catheters according to the present disclosure may cause less trauma to artery walls as compared to conventional atherectomy devices.

### BACKGROUND

### Field

The present disclosure relates to occlusion crossing catheters and methods for using the same.

### Technical Background

Occlusions are blockages of arteries, and a chronic total occlusion (CTO) generally refers to a complete or nearly complete blockage of one or more coronary arteries. Healthy arteries may be flexible and smooth, allowing blood to flow freely through the arteries. However, fatty deposits and calcium can build up on inner artery walls, forming plaque that can partially or completely restrict the flow of blood through the artery. As blood flow is restricted, subjects may experience chest pain, shortness of breath, fatigue, and the like.

In some instances, CTOs can be treated through a “crossing” procedure that is designed to penetrate the occlusion. For example, a catheter can be passed through the artery and can be utilized to break the CTO. In some instances, the catheter may include a catheter tip that transmits ultrasonic vibrations that ablate the plaque, thereby clearing the CTO.

In some circumstances, plaque may be removed from the arteries via an atherectomy procedure. In an atherectomy procedure, a catheter is passed through the artery, and cutting elements attached to the catheter may physically engage plaque within the arteries, causing the plaque to break free from the artery sidewall.

Conventionally, crossing procedures and atherectomy procedures are performed using different devices. Because crossing procedures and atherectomy procedures are performed using different devices, facilities must maintain multiple catheter devices in inventory, thereby increasing operating costs. Further, conventional atherectomy procedures can cause significant trauma to the artery wall. In particular, the physical engagement between cutting elements of the catheter with the plaque and/or artery wall can cause trauma to the artery wall.

Accordingly, a need exists for improved catheters that can perform both a crossing procedure and an atherectomy procedure utilizing a single device. Further, a need exists for improved catheters that reduce the trauma to artery walls during atherectomy procedures.

### BRIEF SUMMARY

Embodiments according to the present disclosure are directed to ultrasonic catheters including vibration wires that are positionable in a retracted position and an extended position. In operation, catheter assemblies according to the present disclosure are operable in an occlusion crossing mode, in which the catheter emits ultrasonic energy with the vibration wires in the retracted position. In embodiments, catheter assemblies according to the present disclosure are also operable in an atherectomy mode, in which the catheter emits ultrasonic energy with the vibration wires in the extended position. With the vibration wires in the extended position, the ultrasonic energy causes the vibration wires to oscillate, and the vibration wires may directly or indirectly engage plaque within an artery, causing the plaque to break free from the artery wall. The oscillation of the vibration wires may be less abrasive than conventional cutting elements used in atherectomy procedures, such that catheters according to the present disclosure may cause less trauma to artery walls as compared to conventional atherectomy devices.

According to one embodiment of the present disclosure, a method for controlling an ultrasonic catheter includes controlling the ultrasonic catheter to operate in an occlusion crossing mode, controlling the ultrasonic catheter to cease operating in the occlusion crossing mode; and controlling the ultrasonic catheter to operate in an atherectomy mode. In the occlusion crossing mode, ultrasonic vibrational energy is supplied from an ultrasonic vibration source to the ultrasonic catheter, while one or more vibration wires of the ultrasonic catheter are in a retracted position. In the atherectomy mode, each of the one or more vibration wires is positioned outward from a distal end of the ultrasonic catheter in an extended position, and ultrasonic vibrational energy is supplied to the one or more vibration wires.

According to another embodiment of the present disclosure, an ultrasonic catheter includes a catheter body extending between a distal end and a proximal end. The catheter body defines a guidewire lumen for receiving a guidewire, which extends through the catheter body. The catheter body also has one or more vibration lumens extending through the catheter body. One or more vibration wires are disposed at least partially within the one or more vibration lumens. The one or more vibration wires are positionable between an extended position and a retracted position. In the extended position, the one or more vibration wires extend further from the catheter body as compared to the retracted position. An ultrasonic vibration source is coupled to the catheter body and configured to impart ultrasonic vibration to the catheter body.

According to another embodiment of the present disclosure, a catheter assembly includes a catheter body. The catheter body includes a guidewire lumen and a guidewire, to be at least partially received in the guidewire lumen, wherein the guidewire extends at least partially through the catheter body. The catheter body also includes one or more extendable vibration wires, wherein the one or more vibration wires extend from a retracted position beyond a distal end of the catheter body in an extended position. The catheter assembly also includes a controller, coupled to the catheter body, with a user input. The catheter body and the controller are both coupled to an ultrasonic power source that vibrates the one or more vibration wires using ultrasonic energy.

Additional features and advantages of the technology disclosed in this disclosure will be set forth in the detailed description which follows, and in part will be readily apparent to those skilled in the art from the description or recognized by practicing the technology as described in this disclosure, including the detailed description which follows, the claims, as well as the appended drawings.

### BRIEF DESCRIPTION OF THE DRAWINGS

The following detailed description of specific embodiments of the present disclosure can be best understood when read in conjunction with the following drawings, where like structure may be indicated with like reference numerals and in which:

FIG. 1 schematically depicts a perspective view of a catheter and a controller, according to one or more embodiments shown and described herein;

FIG. 2 schematically depicts a front axial view of the catheter of FIG. 1, according to one or more embodiments shown and described herein;

FIG. 3 schematically depicts a top view of the catheter of FIG. 1 with one or more vibration wires in an extended position, according to one or more embodiments shown and described herein;

FIG. 4 schematically depicts a top view of the catheter of FIG. 1 with the one or more vibration wires in a retracted position, according to one or more embodiments shown and described herein;

FIG. 5 schematically depicts a flow chart illustrating a method, according to one or more embodiments shown and described herein;

FIG. 6 schematically depicts a longitudinal cross-sectional view of an artery having a total occlusion (TO) or chronic total occlusion (CTO);

FIGS. 7A schematically depicts advancing the catheter of FIG. 1 to the TO or CTO of FIG. 6 with the vibration wires in a retracted position, according to one or more embodiments shown and described herein;

FIG. 7B schematically depicts engaging the catheter of FIG. 1 with the TO or CTO of FIG. 7A with the vibration wires in the retracted position, according to one or more embodiments shown and described herein;

FIG. 8 schematically depicts an exemplary use of the catheter of FIG. 1 removing a TO or CTO with the vibration wires in an extended position, according to one or more embodiments shown and described herein.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

Embodiments described herein are generally directed to ultrasonic catheters including vibration wires that are positionable in a retracted position and an extended position and methods of using the same. In operation, catheter assemblies according to the present disclosure are operable in an occlusion crossing mode, in which the catheter emits ultrasonic energy with the vibration wires in the retracted position. In embodiments, catheter assemblies according to the present disclosure are also operable in an atherectomy mode, in which the catheter emits ultrasonic energy with the vibration wires in the extended position. With the vibration wires in the extended position, the ultrasonic energy causes the vibration wires to oscillate, and the vibration wires may directly or indirectly engage plaque within an artery, causing the plaque to break free from the artery wall. Accordingly, embodiments of the present disclosure provide improved catheter assemblies, which are able to operate in a crossing mode and an atherectomy mode such that only a single piece of equipment is needed. These and other features and embodiments of catheter are disclosed in greater detail herein with reference to the appended figures.

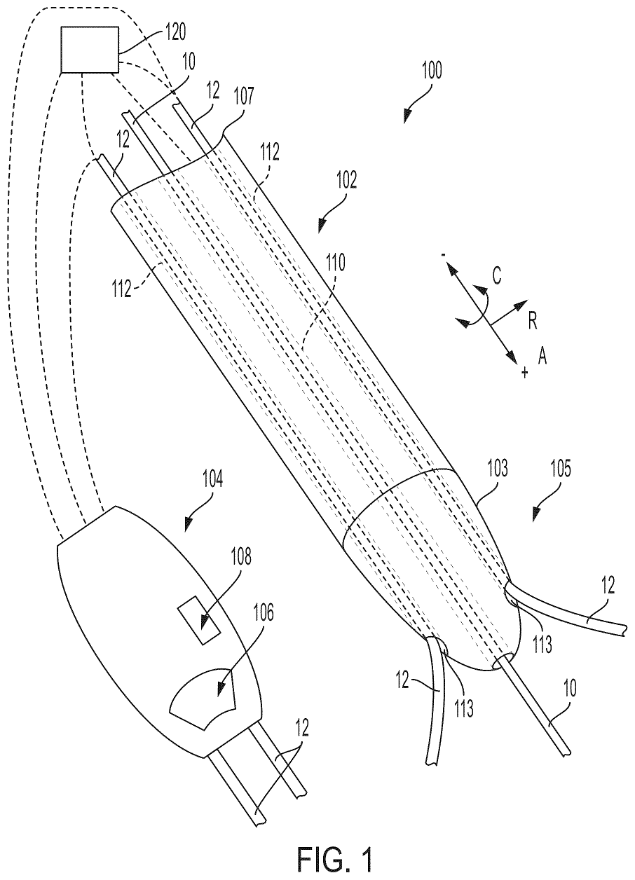

Now referring to FIG. 1, a perspective view of a catheter assembly 100 is schematically depicted. In embodiments, the catheter assembly 100 includes a catheter 102 and a controller 104 coupled (such as communicatively coupled) to the catheter 102. The catheter 102 generally includes a catheter body 103 defining one or more lumens extending through the catheter 102. The catheter body 103 may be formed of any material suitable for advancing through a vessel, such as, but not limited to, polyurethanes, polyamides, fluoropolymers, polyolefins, PVC, polyimides, polyetheretherketone, or the like.

The catheter body 103 extends in an axial direction between a distal end 105 and a proximal end 107. The catheter body 103 defines an axial direction A extending between the proximal end 107 and the distal end 105, a radial direction R that extends perpendicular to the axial direction A, and a circumferential direction C that extends about the axial direction A. In embodiments, the catheter body 103 may be inserted into a lumen of a subject, such as an artery or the like. For example, in some embodiments, a guidewire 10 may be inserted into the lumen of the subject, and the catheter body 103 may be inserted into the lumen of the subject along the guidewire 10. In some embodiments, there may not be a guidewire 10.

In embodiments, the catheter assembly 100 includes one or more vibration wires 12 positioned at least partially within the catheter body 103. The one or more vibration wires 12, in some embodiments, are coupled to the controller 104. The controller 104, in embodiments, is operable to move the one or more vibration wires 12 with respect to the catheter body 103, as described in greater detail herein. In embodiments, the one or more vibration wires 12 are made of shape memory material(s) (e.g., shape memory alloys such as, but not limited to Nitinol, shape, memory polymers, or etc.). However, other materials are contemplated and possible, e.g., titanium, stainless steel, fiber-reinforced thermoplastics, carbon fiber, etc. While in the depicted embodiment of FIG. 1, the catheter assembly 100 includes two vibration wires 12, it should be understood that this is merely an example, and in embodiments according to the present disclosure may include any suitable number of vibration wires 12. For example, in some embodiments, the catheter assembly 100 may include a single vibration wire 12, while in some embodiments, the catheter assembly 100 may include more than two vibration wires 12.

In some embodiments, the catheter assembly 100 includes a vibration source 120. The vibration source 120, in some embodiments, is directly or indirectly coupled to the catheter 102 and/or the one or more vibration wires 12. For example, in some embodiments, the vibration source 120 may be directly coupled to the catheter body 103, e.g., the vibration source 120 may be positioned directly on or within the catheter body 103, and/or the one or more vibration wires 12. In some embodiments, the vibration source 120 is indirectly coupled to the catheter body 103 and/or the one or more vibration wires 12, for example through the guidewire 10 or other intervening component suitable for transmitting vibration from the vibration source 120 to the catheter 102. The vibration source 120 is structurally configured to vibrate the catheter body 103 and/or the one or more vibration wires 12, and in some embodiments, the vibration source 120 is structurally configured to vibrate the catheter body 103 and/or the one or more vibration wires 12 at an ultrasonic frequency. By causing the catheter body 103 and/or the one or more vibration wires 12 to vibrate (e.g., at an ultrasonic frequency), the vibration source 120 may assist the catheter body 103 in crossing an occlusion, as described in greater detail herein. In embodiments, vibration source 120 utilizes an electro-acoustic transducer to convert high frequency electrical voltage to acoustic vibration. This is transmitted to catheter 102 via a sonic connector. In other embodiments, vibration source 120 may include a piezoelectric transducer, configure to operate at various frequencies to provide a desired vibration. For example, the piezoelectric transducer may be made with different sized horns to resonate at a desired operational frequency. Other vibration sources are contemplated and possible.

In embodiments, the vibration source 120 is configurable between an engaged mode and a disengaged mode. In the engaged mode, the vibration source 120 causes the catheter body 103 and/or the one or more vibration wires 12 to vibrate, while in the disengaged mode, the vibration source 120 does not cause the catheter body 103 and/or the one or more vibration wires 12 to vibrate. In some embodiments, the vibration source 120 causes the entire catheter 102 to vibrate in the engaged mode. The vibration source 120, in some embodiments, causes only or substantially only a tip (e.g., the distal end 105) of the catheter 102 to vibrate in the engaged mode, which may in turn cause vibration of the one or more vibration wires 12 when extended as described in greater detail herein.

To operate the vibration source 120 between the engaged mode and the disengaged mode, the controller 104 may be communicatively coupled to the vibration source 120 (e.g., via wired and/or wireless communication) such that the controller 104 may send and/or receive signals from the vibration source 120. That is the controller 104 may be operated to adjust the vibration source 120 between the engaged mode and the disengaged mode. For example, the controller 104 may include a vibration user input 108 structurally configured to adjust the vibration source 120 between the engaged mode and the disengaged mode. The vibration user input 108, in embodiments, may include a push-button, slider, toggle, switch, touch screen, or the like that can be actuated by a user. In embodiments, in response to receiving a user input via operation of the vibration user input 108 (e.g., a user depressing the push-button), the controller 104 sends a signal to the vibration source 120 causing the vibration source 120 to switch between the engaged mode and the disengaged mode. Accordingly, through the vibration user input 108, the vibration source 120 can be selectively engaged and disengaged.

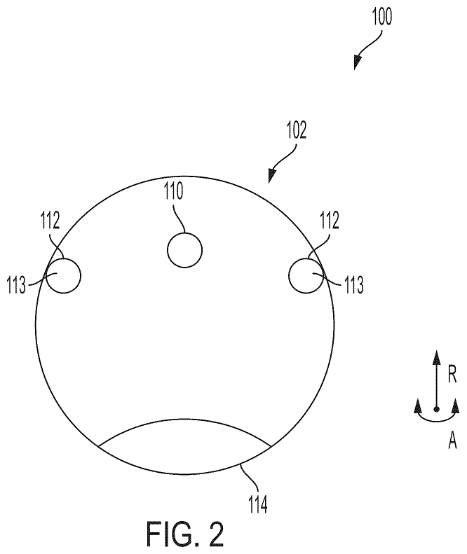

Referring to FIG. 2, an axial view of a distal end of the catheter body 103 is schematically depicted. In embodiments, and as noted above, the catheter body 103 may define one or more lumens extending axially along the catheter body 103 in the axial direction A. For example, in the embodiment depicted in FIG. 2, the catheter body 103 defines a guidewire lumen 110 and one or more vibration wire lumens 112. As shown in FIGS. 1 and 2, the guidewire lumen 110 extends through the catheter body 103 in the axial direction A, such that the guidewire 10 can extend through the catheter body 103 in the axial direction A. The catheter body 103 is movable along the guidewire 10 in the axial direction A, in embodiments. However, the guidewire lumen 110 generally restricts movement of the guidewire 10 (depicted in FIG. 1) in the radial direction R relative to the catheter body 103. In operation, the guidewire 10 may be inserted within a lumen of a subject, such as an artery. The catheter 102 may be moved over the guidewire 10 within the lumen of the subject, thereby assisting in guiding the catheter 102 along the lumen of the subject to a target location (e.g., such as an adjacent TO or TCO).

As noted above, in embodiments, the catheter body 103 defines the one or more vibration wire lumens 112. In the embodiment depicted in FIG. 2, the catheter 102 defines two vibration wire lumens 112, however, it should be understood that this is merely an example. In some embodiments, the catheter body 103 may define a single vibration wire lumen 112, or may define more than two vibration wire lumens 112. For example, there may be a corresponding number of vibration wire lumens to vibration wires 12. In embodiments, the one or more vibration wire lumens 112 are disposed radially offset from guidewire lumen 110. For example, the one or more vibration wire lumens 112 may be positioned along an edge of the catheter body 103. Accordingly, openings 113 of the one or more vibration wire lumens 112 may be open through a sidewall of the catheter body 103, such as depicted in FIG. 1, which may aid in guiding the vibration wires in a splayed orientation when exiting the catheter body 103. The one or more vibration wire lumens 112 extend through the catheter body 103 in the axial direction A, such that the vibration wires 12 can extend through the catheter body 103 in the axial direction A. In embodiments, the one or more vibration wires 12 (depicted in FIG. 1) are positioned at least partially within the one or more vibration wire lumens 112. The one or more vibration wires 12 are movable within the one or more vibration wire lumens 112, such that the one or more vibration wires 12 are movable with respect to the catheter body 103 in the axial direction A. In embodiments, each of the vibration wire lumens 112 may correspond to a single vibration wire 12. In other embodiments, a plurality of vibration wires 12 may be positioned within a single vibration wire lumen 112.

In some embodiments and referring to FIG. 1, the one or more vibration wires 12 are coupled to the controller 104. The controller 104 may be structurally configured to move or slide the one or more vibration wires 12 with respect to the catheter 102. For example, in some embodiments, the controller 104 includes a wire user input 106 that is structurally configured to receive an input from a user. In the embodiment shown in FIG. 1, the wire user input 106 is depicted as a push-button, however it should be understood that this is merely an example, and the wire user input 106 may have any suitable construction to receive an input from a user (e.g., a toggle, switch, slider, touch screen, etc.). In some embodiments, the wire user input 106 is mechanically coupled to the one or more vibration wires 12 such that as a user engages, e.g., pushes, the wire user input 106, the one or more vibration wires 12 move in the axial direction (e.g., in the +A-direction as depicted). In some embodiments, the one or more vibration wires 12 may be collectively actuated. In some embodiments, the one or more vibration wires 12 may be individually actuated. In some embodiments, the wire user input 106 is communicatively coupled to a device such as a motor, a linear actuator, or the like structurally configured to move the one or more vibration wires 12 in the axial direction (e.g., in the +A-direction as depicted). While, in the embodiment depicted in FIG. 1, the wire user input 106 and the vibration user input 108 are depicted as residing on the same controller 104, it should be understood that this is merely an example, and the wire user input 106 and the vibration user input 108 may be located on different controllers.

Referring to FIG. 2, in some embodiments, the catheter body 103 defines a suction lumen 114. The suction lumen 114 extends through the catheter body 103 in the axial direction A, and may be utilized to draw material from a subject's lumen, such as dislodged plaque and debris from an occlusion, as described in greater detail herein. For example, the suction lumen 114 may be communicatively coupled to a vacuum source (e.g., a pump) configured to draw particles, fluid, debris, or the like, from the vessel. In embodiments, the suction lumen 114 is disposed radially offset from a centerline of the catheter body 103, such as along an edge of the catheter body 103, thought other positions are contemplated and possible. In embodiments, a cross sectional area of the suction lumen 114 may be larger than a cross-sectional area of the guidewire lumen 110 and/or the one or more vibration wire lumens 112, so as to be able to draw large particles and/or debris from the vessel during crossing and/or atherectomy procedure.

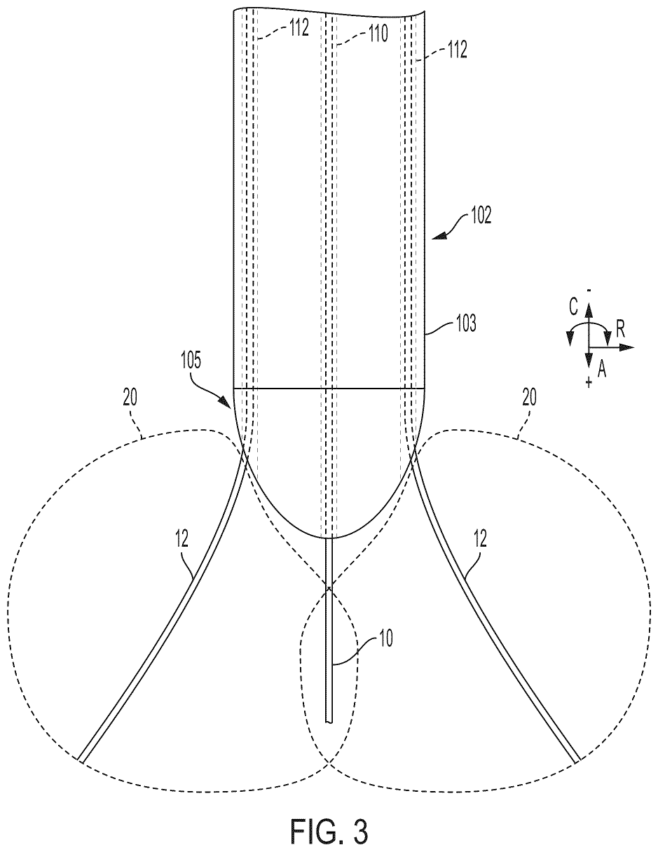

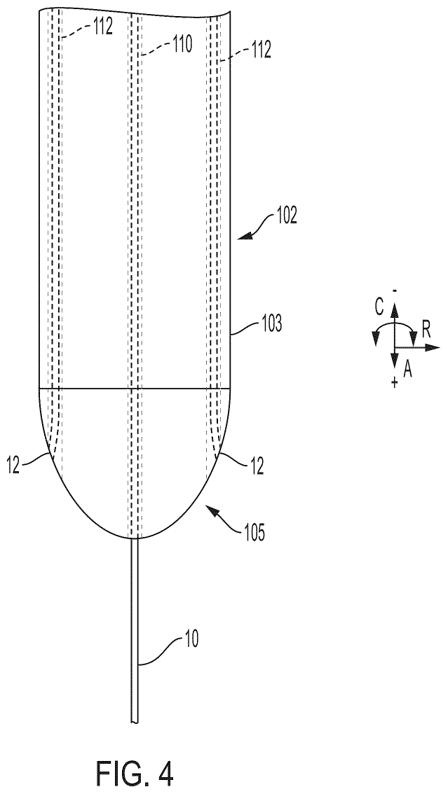

Referring collectively to FIGS. 3 and 4, top views of the catheter body 103 are depicted with the one or more vibration wires 12 in an extended position (FIG. 3) and a retracted position (FIG. 4), respectively. In the extended position shown in FIG. 3, the one or more vibration wires 12 extend further from the catheter body 103 than in the retracted position shown in FIG. 4. In the example depicted in FIG. 4, the distal ends 13 of the one or more vibration wires 12 may be positioned wholly within the distal end 105 of the catheter body 103 in the retracted position.

By contrast, in the extended position, the distal ends 13 of one or more vibration wires 12 extend at least partially outside of the distal end 105 of the catheter body 103 in the extended position such that an operational reach in the radial direction (and/or the longitudinal direction) of the catheter 102 is increased. As noted above, the controller 104 (FIG. 1) may be structurally configured to move the one or more vibration wires 12 between the extended position and the retracted position (e.g., via a push/pull mechanism such as a wheel, lever, motorized linear actuator, or the like). In some embodiments, the one or more vibration wires 12 may be positioned in the retracted position at rest. In some embodiments, the one or more vibration wires 12 may be biased into the retracted position, for example, through a biasing member such as a spring or the like. Where the actuation of the one or more vibration wires 12 from the retracted position to the extended position is motorized, upon receiving input via the wire user input 106 (FIG. 1), the controller 104 (FIG. 1) causes the one or more vibration wires 12 to move from the retracted position to the extended position. By moving the vibration wires 12 between the retracted position and the extended position, the catheter assembly 100 can be operated in an occlusion crossing mode, where vibration wires 12 are retracted or in an atherectomy mode, where the one or more vibration wires 12 are extended.

For example, in some embodiments, the catheter 102 may be passed through a lumen (e.g., a vein or artery) of a subject along the guidewire 10. With the one or more vibration wires 12 in the retracted position, the catheter assembly 100 is operable in the occlusion crossing mode. The occlusion crossing mode, in embodiments, includes supplying vibrational energy (e.g., ultrasonic vibrational energy) to the catheter 102 with the one or more vibration wires 12 in the retracted position as shown in FIG. 4. The vibrational energy (e.g., ultrasonic vibrational energy) can be supplied to the catheter 102 through the vibration source 120 coupled to the catheter 102, as described above. In particular, the vibration user input 108 of the controller 104 may receive input from a user, such as an operator, and the controller 104 directs the vibration source 120 to actuate from the disengaged mode to the engaged mode.

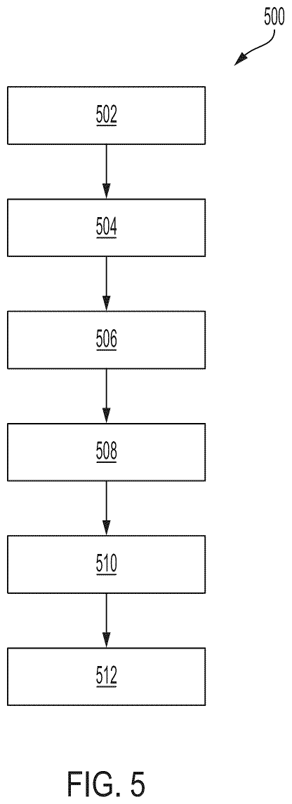

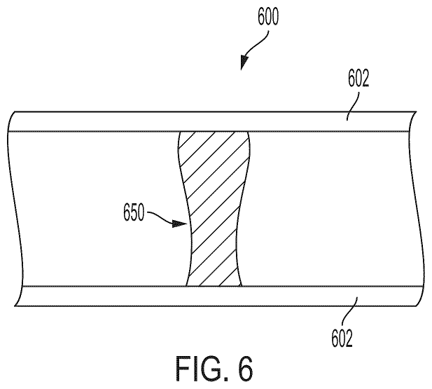

Referring now to FIG. 5, a flow chart illustrating a method 500 of removing or reducing an occlusion using catheter assembly 100 is generally depicted. It is noted that the method 500 may include a greater or fewer number of steps, taken in any order, without departing from the scope of the present disclosure. It is noted that the method 500 illustrated in FIG. 5 may be best understood when reviewed in conjunction with FIGS. 6-8 which generally illustrate alignment and treatment of a blood vessel using the catheter 102 as described herein.

Referring to FIG. 6, a longitudinal cross-sectional view of a vessel 600 (e.g., such as an artery, vein, or other body vessel) is depicted having an occlusion 650, such as a total occlusion (e.g., blocking about 100% of the vessel 600, though occlusions to a lesser degree are contemplated and possible). The occlusion 650 may include atheroma, thrombus, plaque, calcific material, or combinations of thereof.

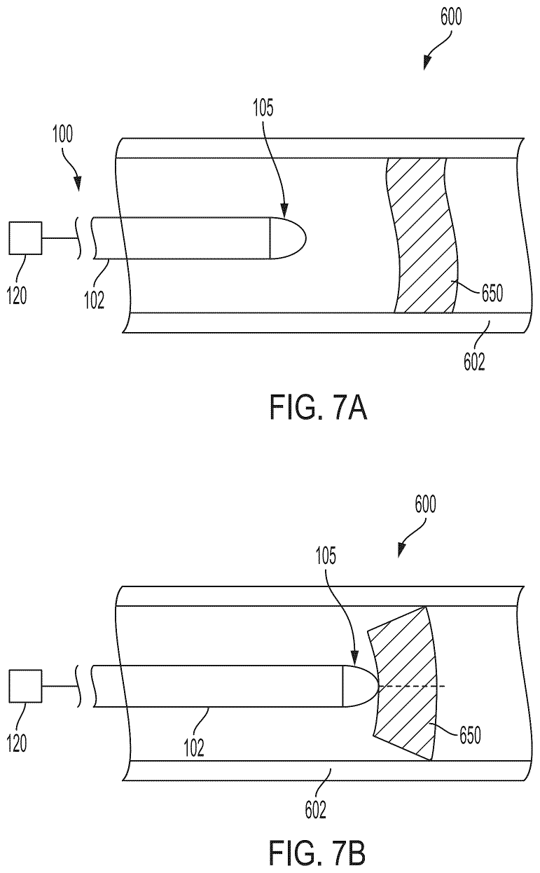

Referring again to FIG. 5, at block 502 the method 500 includes advancing the catheter 102 through a vessel 600 as depicted in FIG. 7A. For example, a user may advance the catheter 102 through the vessel 600 to the occlusion 650 so that the distal end 105 is advanced to the occlusion 650. As shown in FIG. 7A, the catheter 102 can be positioned in the vessel 600 and advanced until the distal end 105 encounters the occlusion 650.

Still referring to FIG. 5, at block 504 the method 500 further includes supplying ultrasonic vibrational energy from an ultrasonic vibration source 120 to the catheter 102. For example, the user may engage vibration source 120 by using vibration user input 108 (FIG. 1) to supply ultrasonic vibrational energy and, as depicted in FIG. 7B, the user may advance the distal end 105 through the occlusion 650 to make or widen an opening in the occlusion 650. During the crossing procedure, particles from the occlusion 650 may become dislodged and may be suctioned into the catheter body 103 via the suction lumen 114, described above.

As noted above, in embodiments, in the engaged mode, the vibration source 120 supplies vibrational energy (e.g., ultrasonic vibrational energy) to the catheter body 103, causing at least a portion of the catheter body 103 to vibrate at an ultrasonic frequency. Without being bound by theory, ultrasonic vibration of the catheter body 103 may assist in breaking up the occlusion 650 within a lumen of a subject (e.g., an artery), thereby allowing the catheter body 103 to pass at least partially through the occlusion 650. In some embodiments, as depicted in FIG. 7B, the catheter body 103 may at least partially engage the occlusion within the lumen of the subject, and vibration of the catheter body 103 causes material forming the occlusion 650, such as plaque and the like, to break free from vessel walls 602 of the subject (e.g., artery walls).

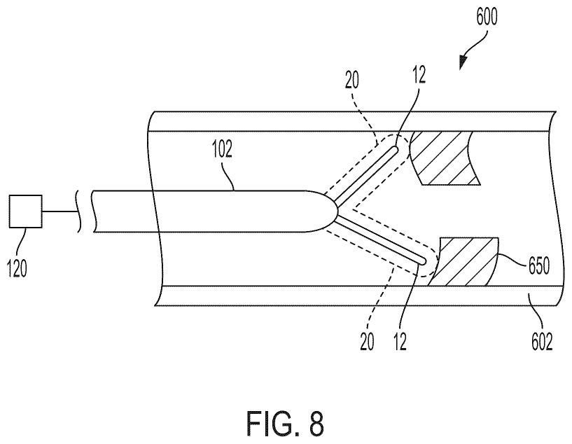

Referring again to FIG. 5, at block 506 the method 500 includes changing the catheter 102 from an occlusion crossing mode wherein the one or more vibration wires 12 are retracted in the catheter body 103 to an atherectomy mode by extending each (or a portion thereof) of the one or more vibration wires 12 into an extended position. For example, using controller 104 (FIG. 1), the user can manipulate the wire user input 106 to move the one or more vibration wires 12 beyond distal end 105 as depicted in FIG. 8. In some embodiments, at least two vibration wires are moved to the extended position in the atherectomy mode.

Referring again to FIG. 5, at block 508 the method 500 includes supplying ultrasonic vibrational energy to the one or more vibration wires 12 in the extended position. For example, the one or more vibration wires 12 may engage remaining parts of occlusion 650 attached to vessel wall 602, as generally depicted in FIG. 8.

In the atherectomy mode, the one or more vibration wires 12 are positioned in the extended position as shown in FIGS. 3 and 8. With the one or more vibration wires 12 in the extended position, vibrational energy (e.g., ultrasonic vibrational energy) is supplied to the catheter 102 via the vibration source 120. Vibration (e.g., ultrasonic vibration) of the catheter 102 causes the one or more vibration wires 12 to vibrate. In particular, the one or more vibration wires 12 are structurally configured to elastically deform upon the application of vibrational energy (e.g., ultrasonic vibrational energy), such that vibration of the catheter body 103 causes the one or more vibration wires 12 to vibrate. In this way, the vibration source 120 may indirectly supply vibrational energy to the one or more vibration wires 12 (e.g., via the catheter 102). However, in other embodiments and as described herein, the one or more vibration wires 12 may be directly coupled to the vibration source 120, such that the one or more vibration wires 12 are directly vibrated via the vibration source.

As shown in FIG. 8, the one or more vibration wires 12 may engage the occlusion 650 in the extended position. In embodiments, the one or more vibration wires 12 may vibrate or move in non-periodic or random manner within a vibrational envelope 20, generating a larger area of vibration around the catheter. In embodiments, the one or more vibration wires 12 may engage material, such as plaque, within the subject's lumen as the vibration wires 12 move within their vibrational envelopes 20. The one or more vibration wires 12 may be formed of wires and/or abrasive materials that may fracture the plaque, thereby breaking up the occlusion 650 and either widening or forming an opening through the occlusion to restore flow. Because the one or more vibration wires 12 may move in a non-periodic or random manner, the one or more vibration wires 12 may not generally maintain constant contact with the walls of the subject's lumen (e.g., the artery walls) or with material along the walls of the subject's lumen (e.g., plaque along the artery walls).

Conventional atherectomy devices generally include cutting devices, such as blades, burrs, and the like that are in constant or near constant contact with the walls of the subject's lumen (e.g., the artery walls) or with the material along the walls of the subject's lumen (e.g., plaque along the artery walls). Because the one or more vibration wires 12 intermittently contact the walls of the subject's lumen (e.g., the artery walls) and/or the material, the one or more vibration wires 12 may cause less unwanted contact to the walls of the subject's lumen (e.g., the artery walls) than conventional atherectomy cutting devices.

Still referring to FIG. 5, at block 510 the method 500 may include drawing material into the suction lumen 114 defined by the ultrasonic catheter. For example, the material broken free from the walls of the lumen (e.g., plaque and the like), in some embodiments, is drawn through the suction lumen 114 (FIG. 2), thereby removing the material from the lumen of the subject.

Referring again to FIG. 5, at block 512 the method 500 may include includes changing the catheter 102 from atherectomy mode wherein the one or more vibration wires 12 are in an extended position back to an occlusion crossing mode, where in the vibration wires are in a retracted position. For example, using controller 104 (FIG. 1), the user can manipulate the wire user input 106 to move the one or more vibration wires 12 back into a retracted position as needed or desired. In this way, through selectively moving the one or more vibration wires 12 between the extended position and the retracted position, catheter assemblies according to the present disclosure may be utilized in both crossing procedures and atherectomy procedures.

In some embodiments, subsequent to operating the catheter assembly 100 in the atherectomy mode, the catheter 102 may be withdrawn from the subject's lumen along the guidewire 10.

Embodiments can be described with reference to the following numerical clause:

1. A method of controlling an ultrasonic catheter, the method comprising: controlling the ultrasonic catheter to operate in an occlusion crossing mode, the occlusion crossing mode comprising: supplying ultrasonic vibrational energy from an ultrasonic vibration source to the ultrasonic catheter, wherein one or more vibration wires of the ultrasonic catheter are in a retracted position in the occlusion crossing mode; controlling the ultrasonic catheter to cease operating in the occlusion crossing mode; and controlling the ultrasonic catheter to operate in an atherectomy mode, the atherectomy mode comprising: positioning each of the one or more vibration wires in an extended position, wherein each of the one or more vibration wires extend further outward from a distal end of the ultrasonic catheter in the extended position as compared to the retracted position; and controlling the ultrasonic catheter to supply ultrasonic vibrational energy to the one or more vibration wires with the one or more vibration wires in the extended position.

2. The method of the preceding clause, wherein the ultrasonic catheter is controlled such that ultrasonic vibrational energy is indirectly supplied to the one or more vibration wires.

3. The method of any preceding clause, wherein the ultrasonic catheter is controlled such that the one or more vibration wires are positioned within the distal end of the ultrasonic catheter in the retracted position.

4. The method of any preceding clause, further comprising controlling the ultrasonic catheter to draw material into a suction lumen defined by the ultrasonic catheter.

5. The method of any preceding clause, further comprising, controlling the ultrasonic catheter to, subsequent to operating the ultrasonic catheter in the atherectomy mode, move the one or more vibration wires into the retracted position.

6. The method of any preceding clause, further comprising controlling the ultrasonic catheter to engage an occlusion with the one or more vibration wires.

7. The method of any preceding clause, wherein the ultrasonic catheter is controlled such that positioning each of the one or more vibration wires in the extended position comprises positioning at least two vibration wires in the extended position.

8. The method of any preceding clause, further comprising controlling the ultrasonic catheter to move the ultrasonic catheter within a lumen by moving the ultrasonic catheter along a guidewire.

9. An ultrasonic catheter comprising: a catheter body extending between a distal end and a proximal end, the catheter body defining: a guidewire lumen for receiving a guidewire extending through the catheter body; and one or more vibration wire lumens extending through the catheter body; one or more vibration wires positioned at least partially within the one or more vibration wire lumens, wherein the one or more vibration wires are positionable between an extended position and a retracted position, wherein the one or more vibration wires extend (protrude, project) further from the catheter body in the extended position as compared to the retracted position; and an ultrasonic vibration source coupled to the catheter body and configured to impart ultrasonic vibration to the catheter body.

10. The ultrasonic catheter of the preceding clause, wherein the catheter body further defines a suction lumen extending through the catheter body.

11. The ultrasonic catheter of any preceding clause 9 to 10, wherein the one or more vibration wires comprises at least two vibration wires.

12. The ultrasonic catheter of any preceding clause 9 to 11, wherein the one or more vibration wires are positioned within the catheter body when in the retracted position.

13. The ultrasonic catheter of any preceding clause 9 to 12, further comprising a controller, the controller comprising a user input structurally configured to move the one or more vibration wires between the extended position and the retracted position.

14. The ultrasonic catheter of any preceding clause 9 to 13, further comprising a controller, the controller comprising a user input structurally configured to move the ultrasonic vibration source between a disengaged mode, and an engaged mode, in which the ultrasonic vibration source causes the catheter body to vibrate.

15. A catheter assembly comprising: a catheter, comprising a catheter body; a guidewire lumen and a guidewire to be at least partially received in the guidewire lumen, wherein the guidewire is to extend at least partially through the catheter body; one or more vibration wires, wherein the catheter assembly is configured such that the one or more vibration wires are configured to extend from a retracted position beyond a distal end of the catheter body in an extended position; a controller/control unit for controlling an amount of ultrasonic vibrational energy to be imparted to the catheter body comprising a user input, wherein the controller/control unit is coupled to the catheter body; and an ultrasonic vibration source coupled to the catheter body and the controller/control unit, wherein the catheter assembly is configured such that the ultrasonic vibration source vibrates the one or more vibration wires using an amount of ultrasonic vibrational energy.

16. The catheter assembly of the preceding clause wherein the catheter assembly and/or the ultrasonic vibration source is configured such that the ultrasonic vibrational energy is indirectly supplied to the one or more vibration wires.

17. The catheter assembly of any preceding clause 15 to 16, further comprising a suction lumen defined by the catheter body and/or wherein the catheter body comprises a suction lumen.

18. The catheter assembly of any preceding clause 15 to 17, wherein the catheter assembly and/or user input is structurally configured to move the one or more vibration wires to the extended position.

19. The catheter assembly of any preceding clause 15 to 18, wherein the catheter assembly and/or user input is structurally configured to engage or disengage the ultrasonic vibration source.

20. The catheter assembly of any preceding clause 15 to 19, wherein the catheter assembly is configured such that the ultrasonic vibrational energy can be applied to the one or more vibration wires in the extended position and the retracted position.

The ultrasonic catheter of clauses 9 to 14 may be combined with the catheter assembly of any of clauses 15 to 20 and may be used for the method of clauses 1 to 8. Also the catheter assembly of clauses 15 to 20 and may be used for the method of clauses 1 to 8.

Accordingly, it should now be understood that embodiments described herein are directed to ultrasonic catheters including vibration wires that are positionable in a retracted position and an extended position. In operation, catheter assemblies according to the present disclosure are operable in an occlusion crossing mode, in which the catheter emits ultrasonic energy with the vibration wires in the retracted position. In embodiments, catheter assemblies according to the present disclosure are also operable in an atherectomy mode, in which the catheter emits ultrasonic energy with the vibration wires in the extended position. With the vibration wires in the extended position, the ultrasonic energy causes the vibration wires to oscillate, and the vibration wires may directly or indirectly engage plaque within an artery, causing the plaque to break free from the artery wall.

Having described the subject matter of the present disclosure in detail and by reference to specific embodiments, it is noted that the various details described in this disclosure should not be taken to imply that these details relate to elements that are essential components of the various embodiments described in this disclosure, even in cases where a particular element is illustrated in each of the drawings that accompany the present description. Rather, the appended claims should be taken as the sole representation of the breadth of the present disclosure and the corresponding scope of the various embodiments described in this disclosure. Further, it should be apparent to those skilled in the art that various modifications and variations can be made to the described embodiments without departing from the spirit and scope of the claimed subject matter. Thus it is intended that the specification cover the modifications and variations of the various described embodiments provided such modification and variations come within the scope of the appended claims and their equivalents.

It is noted that recitations herein of a component of the present disclosure being “structurally configured” in a particular way, to embody a particular property, or to function in a particular manner, are structural recitations, as opposed to recitations of intended use. More specifically, the references herein to the manner in which a component is “structurally configured” denotes an existing physical condition of the component and, as such, is to be taken as a definite recitation of the structural characteristics of the component.

It is noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

For the purposes of describing and defining the present invention it is noted that the terms “substantially” and “about” are utilized herein to represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

1. A method of controlling an ultrasonic catheter, the method comprising:
controlling the ultrasonic catheter to operate in an occlusion crossing mode, the occlusion crossing mode comprising:
supplying ultrasonic vibrational energy from an ultrasonic vibration source to the ultrasonic catheter, wherein one or more vibration wires of the ultrasonic catheter are in a retracted position in the occlusion crossing mode;
controlling the ultrasonic catheter to cease operating the ultrasonic catheter in the occlusion crossing mode; and controlling the ultrasonic catheter to operate in an atherectomy mode, the atherectomy mode comprising:
positioning each of the one or more vibration wires in an extended position, wherein each of the one or more vibration wires extend further outward from a distal end of the ultrasonic catheter in the extended position as compared to the retracted position; and
controlling the ultrasonic catheter to supply ultrasonic vibrational energy to the one or more vibration wires with the one or more vibration wires in the extended position.

2. The method of claim 1, wherein the ultrasonic catheter is controlled such that the ultrasonic vibrational energy is indirectly supplied to the one or more vibration wires.

3. The method of claim 1, wherein the ultrasonic catheter is controlled such that the one or more vibration wires are positioned within the distal end of the ultrasonic catheter in the retracted position.

4. The method of claim 1, further comprising controlling the ultrasonic catheter to draw material into a suction lumen defined by the ultrasonic catheter.

5. The method of claim 1, further comprising, controlling the ultrasonic catheter to, subsequent to controlling the ultrasonic catheter to operate in the atherectomy mode, move the one or more vibration wires into the retracted position.

6. The method of claim 1, further comprising controlling the ultrasonic catheter to engage an occlusion with the one or more vibration wires.

7. The method of claim 1, wherein the ultrasonic catheter is controlled such that positioning each of the one or more vibration wires in the extended position comprises positioning at least two vibration wires in the extended position.

8. The method of claim 1, further comprising controlling the ultrasonic catheter to move within a lumen by moving the ultrasonic catheter along a guidewire.

9. An ultrasonic catheter comprising:
a catheter body extending between a distal end and a proximal end, the catheter body defining:
a guidewire lumen for receiving a guidewire extending through the catheter body; and
one or more vibration wire lumens extending through the catheter body;
one or more vibration wires positioned at least partially within the one or more vibration wire lumens, wherein the one or more vibration wires are positionable between an extended position and a retracted position, wherein the one or more vibration wires extend further from the catheter body in the extended position as compared to the retracted position; and
an ultrasonic vibration source coupled to the catheter body and configured to impart ultrasonic vibration to the catheter body.

10. The ultrasonic catheter of claim 9, wherein the catheter body further defines a suction lumen extending through the catheter body.

11. The ultrasonic catheter of claim 9, wherein the one or more vibration wires comprises at least two vibration wires.

12. The ultrasonic catheter of claim 9, wherein the one or more vibration wires are positioned within the catheter body when in the retracted position.

13. The ultrasonic catheter of claim 9, further comprising a controller, the controller comprising a user input structurally configured to move the one or more vibration wires between the extended position and the retracted position.

14. The ultrasonic catheter of claim 9, further comprising a controller, the controller comprising a user input structurally configured to move the ultrasonic vibration source between a disengaged mode, and an engaged mode, in which the ultrasonic vibration source causes the catheter body to vibrate.

15. A catheter assembly comprising:
a catheter, comprising
a catheter body;
a guidewire lumen and a guidewire, to be at least partially received in the guidewire lumen, wherein the guidewire extends at least partially through the catheter body;
one or more vibration wires, wherein the catheter assembly is configured such that the one or more vibration wires are configured to extend from a retracted position beyond a distal end of the catheter body in an extended position;
a controller for controlling an amount of ultrasonic vibrational energy to be imparted to the catheter body comprising a user input, wherein the controller is coupled to the catheter body; and
an ultrasonic vibration source coupled to the catheter body and the controller, wherein the catheter assembly is configured such that the ultrasonic vibration source vibrates the one or more vibration wires using an amount of ultrasonic vibrational energy.

16. The catheter assembly of claim 15 wherein the catheter assembly is configured such that the ultrasonic vibrational energy is indirectly supplied to the one or more vibration wires.

17. The catheter assembly of claim 15, further comprising a suction lumen defined by the catheter body.

18. The catheter assembly of claim 15, wherein the user input is structurally configured to move the one or more vibration wires to the extended position.

19. The catheter assembly of claim 15, wherein the user input is structurally configured to engage or disengage the ultrasonic vibration source.

20. The catheter assembly of claim 15, wherein the catheter assembly is configured such that the ultrasonic vibrational energy can be applied to the one or more vibration wires in the extended position and the retracted position.
